(19) United States
(12) Patent Application Publication
(10) Pub. No.: US 2004/0176585 A1
(43) Pub. Date: Sep. 9, 2004

(54) Method and system for determining optimal sequences

(76) Inventor: Richard J. Carter, Los Altos, CA (US)

Correspondence Address: HEWLETT-PACKARD DEVELOPMENT COMPANY, Intellectual Property Administration, P.O. Box 272400, Fort Collins, CO, 80527-2400, US

(21) Appl. No.: 10/375,858

(22) Filed: Feb. 27, 2003

(51) Int. Cl. G06F 19/00; G01N 33/48; G01N 33/50; C07H 21/04

(52) U.S. Cl. 536/24.3; 702/20

(57) ABSTRACT

A method and system for searching for an optimal sequence of subunits from among possible sequences of subunits. A described embodiment provides for evaluating each possible subunit sequence of a biopolymer with respect to a mass spectrum produced by ionizing and fragmenting the mass-tagged biopolymer in order to determine the most probable subunit sequence of the biopolymer. However, unlike in the currently practiced, permutations-based methods, an efficient combinations-based search is used to hierarchically partition all possible subunit sequences, and to assign maximum subunit-sequence rankings to the partitions, in order to computationally efficiently extract the most probable subunit sequence.

### TECHNICAL FIELD

The present invention relates to computational methods and analytical systems employing computational methods for determining optimal subunit sequences with respect to a measurable, calculable, or estimable quantity or characteristic, and, in particular, to a more efficient method for determining the optimal subunit sequence or sequences including, in one embodiment, the most probable terminal sequence of a biopolymer from mass-spectrum data.

### BACKGROUND OF THE INVENTION

Characterization of biopolymers is a fundamental task in modern biology, structural genomics, proteomics, molecular biology, and a host of other interrelated fields. Biopolymers are the fundamental information-containing, energy-containing, and structural components of living organisms. Many different types of biopolymers are found in living organisms, including ribonucleic acids (“RNA”), deoxyribonucleic acids (“DNA”), proteins, glycoproteins and polysaccharides. Certain of the biopolymers, including DNA, RNA, and proteins, are synthesized as linear sequences of monomer subunits, also referred to as “residues,” linked together via enzymatically catalyzed condensation reactions. Other biopolymers, including polysaccharides, have complex, acyclic-graph-like structures.

FIGS. 1A-B show representations of the chemical structures of DNA and proteins, respectively. As shown in FIG. 1A, the familiar DNA-double-helix molecule pair comprises two linear DNA strands 102 and 103 twisted together in a double helix. A single DNA molecule is a linear sequence of nucleotide monomers linked together through phosphate groups. The sequence of pyrimidine and purine bases of the nucleotide monomers contains genetic information. In FIG. 1A, a portion of the DNA polymer backbone includes a first phosphate 104 linked to a deoxyribose moiety 105, in turn linked to a second phosphate group 106, with a purine side chain 107 linked to the deoxyribose moiety 105. The purine side group 107, along with the deoxyribose moiety 105 and one of the two phosphates 104 and 106 together comprise a monomer subunit of the DNA biopolymer. In proteins, as shown in FIG. 1B, amino-acid-subunits are linked together in a linear chain 110, with each amino-acid-subunit, such as the phenylalanine subunit 112 shown within dashed lines in FIG. 1B, contributing two carbon atoms 114 and 115 and a nitrogen atom 116 to the backbone, as well as a side chain moiety 118.

One embodiment of the present invention concerns determination of the sequence of chemical identities of monomer subunits of biopolymers, such as DNA and proteins, using mass spectrometry. For the purposes of describing the present invention, linear biopolymers, including DNA and proteins, can be thought of as a backbone to which side groups are attached at regular internals. Moreover, both the backbone and side groups can be considered to comprise a number of discrete building blocks having characteristic masses. FIG. 2 illustrates an abstract view of biopolymers suitable for description of the present invention. In FIG. 2, the backbone of the biopolymer 202 is a regularly structured sequence of building blocks, such as building block 204. Each building block has a characteristic mass, such as the mass 14 for building block 204. Note that integral values for atomic masses are used in the figures and examples, for clarity of illustration, although, for various reasons, atomic masses are generally not integral values. Side groups, including side groups 206-213 in FIG. 2, are attached to the backbone 202 at regularly spaced intervals. FIG. 2 shows an abstract representation of a portion of the sequence of amino-acid monomers linked together to form a protein. The side groups 206-213 are amino-acid side groups, and the backbone is composed of a series of repeating building blocks 214 that include a nitrogen atom 216, an α-carbon atom 217, and a carbonyl moiety, including a carbon atom 218 and oxygen atom 219. Note that the chemical bonds and spatial orientation of side-group atoms is not reflected in the abstract representation, and is generally unnecessary for sequence analysis. FIG. 3 shows the building-block components of side groups. Each side group, such as side group 302, is composed of building blocks, and each side group therefore has a characteristic mass. In general, determining the sequence of subunits of a biopolymer refers to a process by which the chemical identities of the side chains attached to a liner polymer are determined, equivalent to determining the chemical identities of the monomer subunits from which the polymer was sequentially synthesized.

FIGS. 4A-F, 5, and 6 together illustrate analysis of terminal sequences of biopolymers using mass spectrometry. FIGS. 4A-F illustrate mass tagging of the terminus of a biopolymer followed by ionization and fragmentation of the biopolymer. As shown in FIG. 4A, a mass tag 402 can be chemically attached to the end of a biopolymer backbone 404. In general, the mass tag is a relatively heavy chemical entity, such as a metal cluster or heavy-element-containing organic compound, with a mass selected for easy detection in a mass spectrum. Once the mass tag has been chemically attached to the end of the biopolymer, the biopolymer may be ionized and vaporized, for example via electrospray ionization or matrix-assisted laser desorption/ionization (“MALDI”). Fragmentation of the biopolymer is subsequently achieved via the nozzle potential of the mass spectrometer, collisionally induced dissociation (“CID”), or by other means. In general, the biopolymer backbone may be broken in many possible ways, with fragmentation at certain points within the backbone more probable than at other points. FIG. 4B shows three different potential sites for fragmentation of the example mass-tagged biopolymer shown in FIG. 4A that produce a four-subunit, or four-residue, fragment of the biopolymer. If the biopolymer fragments between two backbone carbon atoms 406, the mass-tag fragment shown in FIG. 4C is produced, with an approximate characteristic mass of M+467, where M is the mass of the mass tag. When the biopolymer is fragmented between the carbonyl carbon and the nitrogen atom 407, the four-subunit fragment shown in FIG. 4D is produced, with an approximate characteristic mass of 495+M. When the biopolymer is fragmented between the nitrogen and α-carbon of the next monomer 408, the four-subunit fragment shown in FIG. 4E is produced, with an approximate characteristic mass of 510+M. Moreover, additional building blocks may be lost during ionization and fragmentation of the biopolymer. For example, as shown in FIG. 4F, the four-subunit fragment shown in FIG. 4C may additionally lose a hydrogen atom from the first side group 410 to produce a fourth type of four-subunit fragment having an approximate characteristic mass of 466+M. Of course, if a proton is lost, a negative charge is added to the ion. In the case that the ion was already singly negatively charged, the negative charge is doubled, and the expected position of the doubly negatively charged fragment in the mass spectrum would be quite different from that of a singly negatively charged fragment of the same mass.

FIG. 5 shows the example mass-tagged biopolymer of FIG. 4A with indications of possible fragmentation locations. Even the small, eight-subunit biopolymer shown in FIG. 5 can produce hundreds of different fragments with different characteristic masses. For example, more than one cleavage may take place, producing different types of internal fragments lacking the mass tag. Different atoms from subgroups may be lost during the ionization process, indicated by the “x” markings, such as “x” marking 502 in FIG. 5. During mass-spectrum analysis of a biopolymer, in which ionized and vaporized fragments are accelerated through an electric field to a target detector, all possible combinations of fragmentation may occur to produce a large number of different types of fragments with characteristic masses. FIG. 6 shows a hypothetical mass spectrum that may result from analysis of fragmentation products of the example biopolymer shown in FIG. 4A. In general, mass-spectrometry analysis of a mass-tagged biopolymer produces a plot, as shown in FIG. 6, with peaks, such as peak 602, occurring along the x-axis 604. The peaks are positioned along the x-axis according to the mass/charge ratio of a fragmentation product that produced the peak. The amount of each type of fragmentation product produced is reflected in the height of the peak, as measured along the y-axis 606. Assuming that fragmentation most likely occurs between the backbone carbonyl groups and nitrogen atoms (407 in FIG. 4B, for example), and further assuming that single-backbone-breakage fragmentation products are generally most probable, then the highest peaks in the hypothetical mass spectrum 602-605 and 607-610 may correspond to 1-, 2-, 3-, 4-, 5-, 6-, 7-, and 8-subunit single-breakage fragmentation products of the example biopolymer shown in FIG. 4A. However, in general, mass-spectrum data is not so easily interpreted. A mass spectrum produced from a biopolymer may contain hundreds or thousands of major and minor peaks, with internal fragmentation fragments lacking mass tags interspersed with mass-tagged single-breakage fragments. In addition, any given fragment type may exist at multiple mass/charge points of the mass spectrum due to the possible inclusion of naturally occurring atomic isotopes in the fragment.

Mass-spectrum analysis of biopolymer sequences is attractive because, unlike tedious chemical sequencing, it can be theoretically carried out extremely rapidly. A large biopolymer may be chemically cleaved and/or enzymatically digested, the cleavage and/or digest products may be separated by chromatography or electrophoresis and mass-tagged, and the mass-tagged digest products then analyzed by mass spectrometry to produce sequences for the biopolymer digest products. If the enzymatic digestion is performed multiple times with digestive enzymes having different biopolymer cleavage properties, the sequences of overlapping biopolymer digest products can then be computationally reassembled into the full biopolymer sequence. Alternatively, an intact biopolymer can be mass-tagged and subject to ionization and fragmentation to produce a series of terminal sequences that can lead to a unique or partial identification of the biopolymer from a database of known biopolymers. The term “biopolymer” is used below to refer to both large intact biopolymers and the products that result from chemical cleavage or enzymatic digestion. The term “protein” is used below to refer to both large intact proteins and any polypeptide that might result from the enzymatic digestion or chemical cleavage of a protein.

The computational analysis of a mass spectrum to determine the sequence of a mass-tagged biopolymer from which the mass spectrum was produced is a computationally complex process. Designers and manufactures of analytical instrumentation, users of analytical instrumentation, and experimenters have all recognized the need for more computationally efficient methods for extracting biopolymer subunit sequences from mass spectra produced by ionizing, vaporizing and fragmenting mass-tagged biopolymers.

### SUMMARY OF THE INVENTION

Various embodiments of the present invention provide methods and systems for searching for an optimal sequence of subunits from among possible sequences of subunits. In one embodiment of the present invention, each possible subunit sequence of a biopolymer is evaluated with respect to a mass spectrum produced by ionizing and fragmenting the mass-tagged biopolymer in order to determine the most probable subunit sequence of the biopolymer. However, unlike in the currently practiced, permutations-based methods, an efficient combinations-based search is used to hierarchically partition all possible subunit sequences, and to assign maximum subunit-sequence rankings to the partitions, in order to computationally efficiently extract the most probable subunit sequence.

### BRIEF DESCRIPTION OF THE DRAWINGS

FIGS. 1A-B show representations of the chemical structures of DNA and proteins, respectively.

FIG. 2 illustrates an abstract view of biopolymers suitable for description of the present invention.

FIG. 3 shows the building-block components of side groups.

FIGS. 4A-F illustrate mass tagging of the terminus of a biopolymer followed by ionization and fragmentation of the biopolymer.

FIG. 5 shows the example mass-tagged biopolymer of FIG. 4A with indications of possible fragmentation locations.

FIG. 6 shows a hypothetical mass spectrum that may result from analysis of fragmentation and ionization products of the example biopolymer shown in FIG. 4A.

FIG. 7 illustrates a currently available process by which a greatest ranked 3-subunit sequence of a biopolymer is determined from the mass spectrum produced by mass tagging the biopolymer and subjecting the biopolymer to mass spectrometry.

FIG. 8 illustrates calculation of the maximum sequence ranking of a set of sequences representing the permutations of a particular combination of subunit types according to one embodiment of the present invention.

FIG. 9 illustrates the combination approach to calculating the most probable biopolymer sequence from a mass spectrum that represents one embodiment of the present invention.

FIG. 10 shows two data structures that enable one embodiment of the method of the present invention to be carried out in a straightforward manner.

FIG. 11 is a flow-control diagram of a routine “find_optimal_sequence,” an embodiment of the present invention.

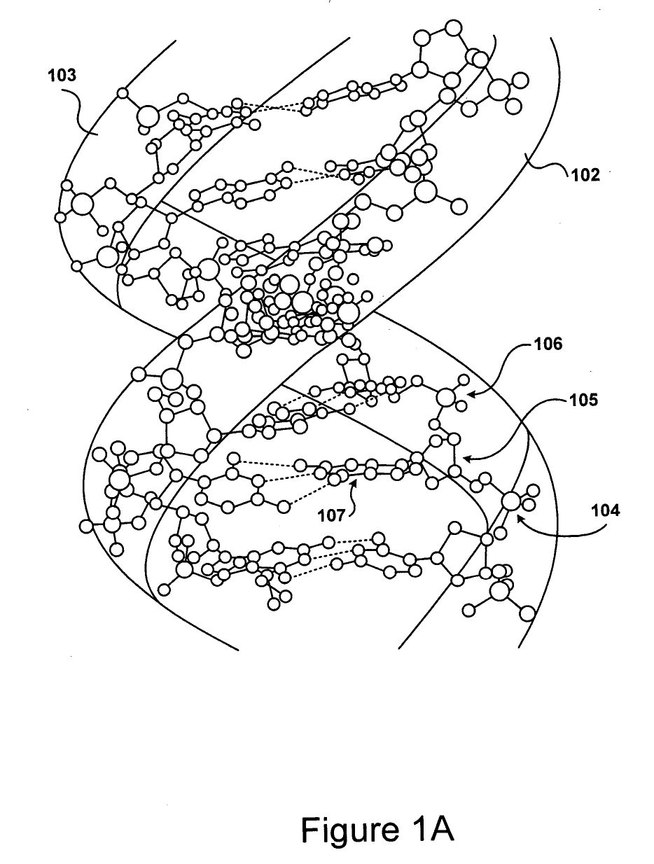

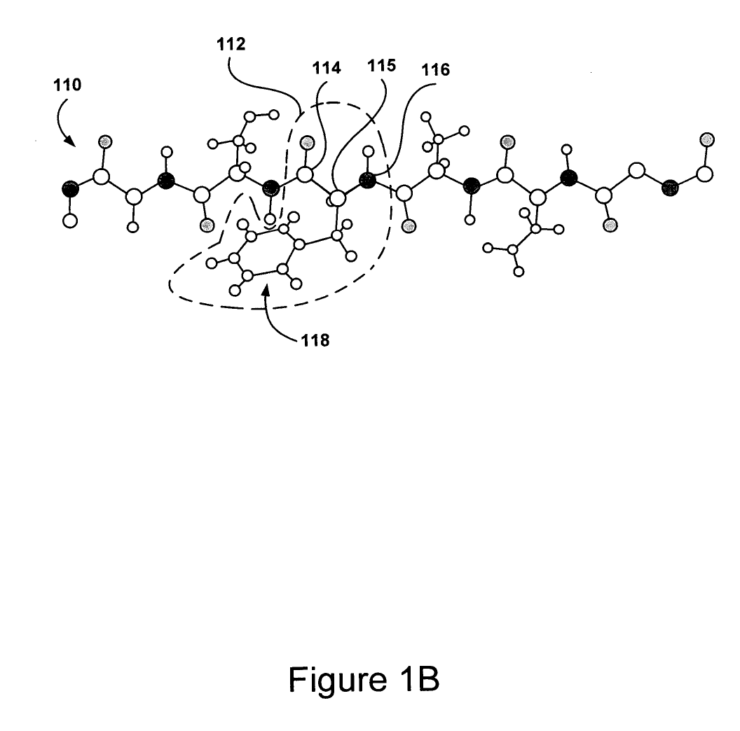

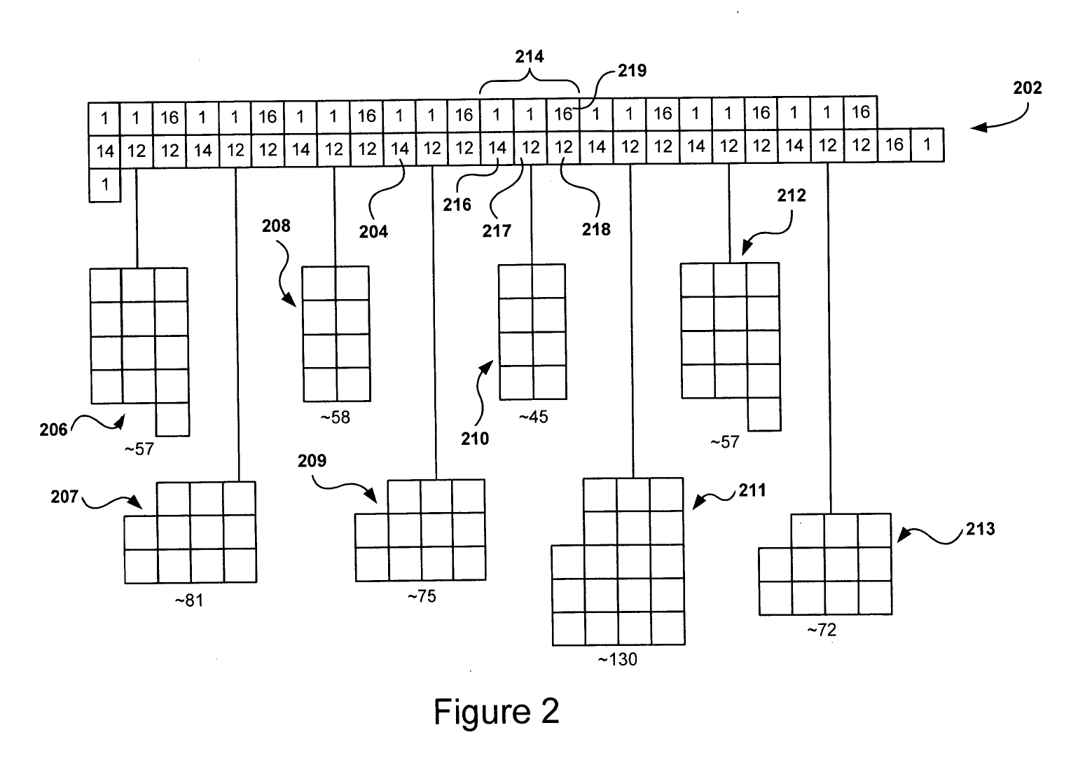

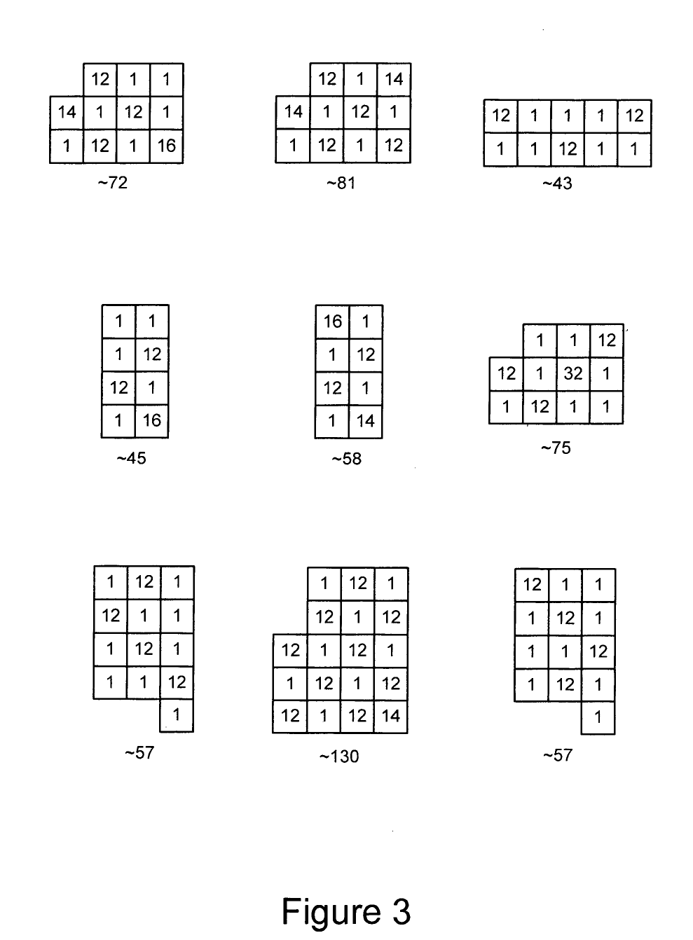

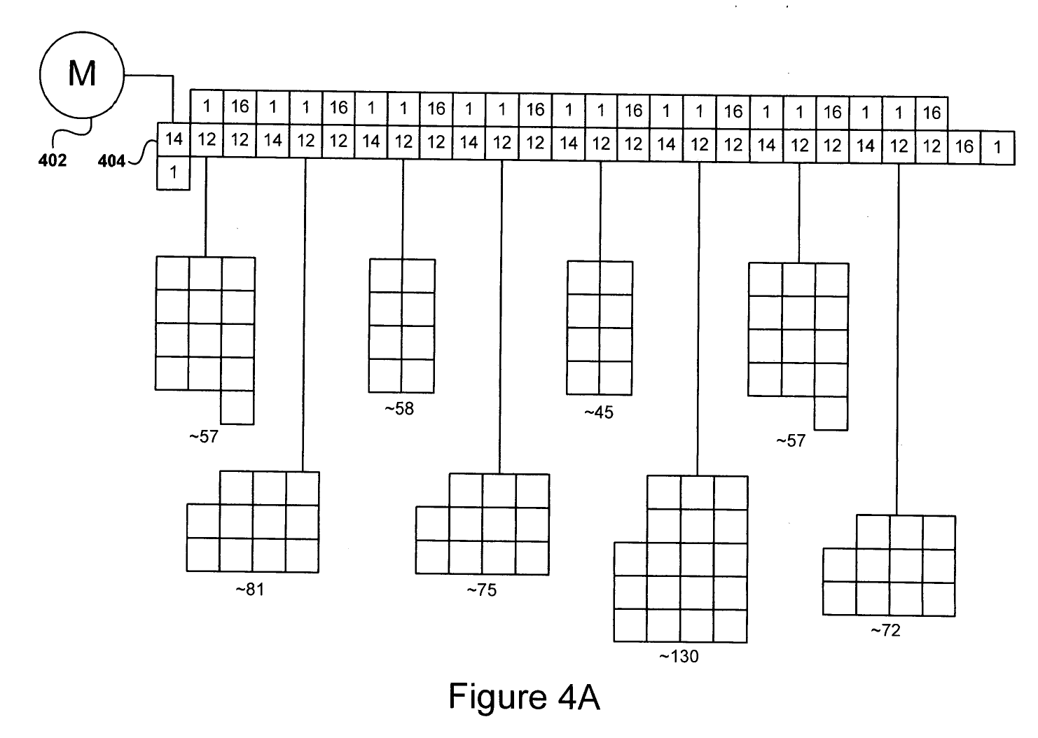

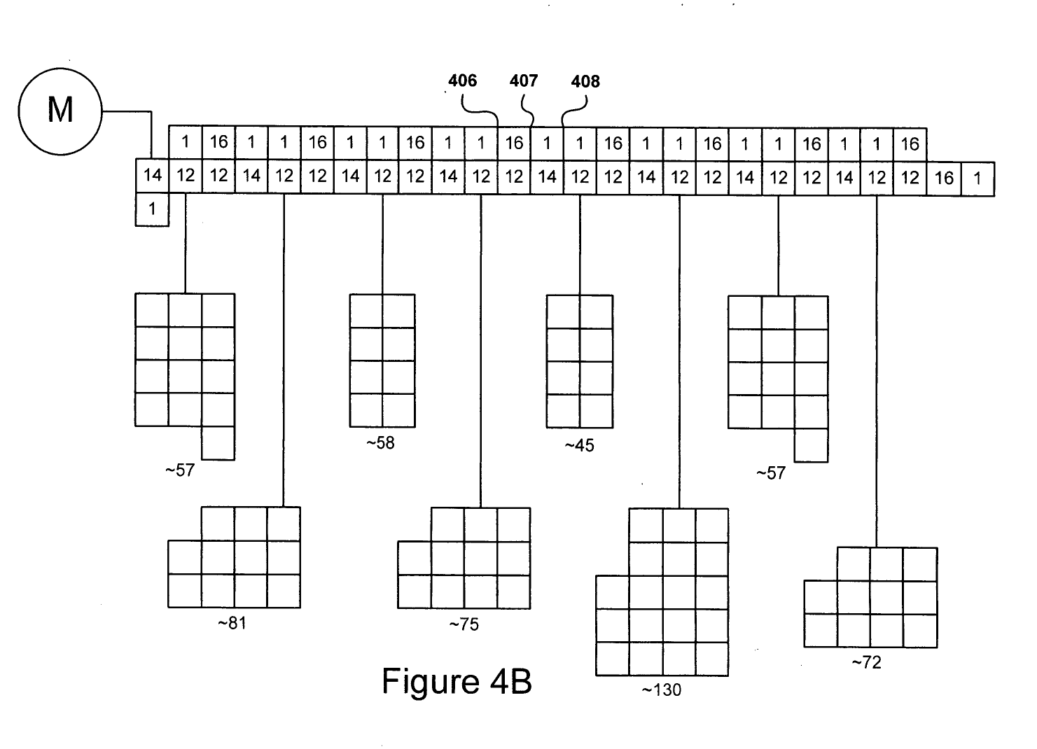

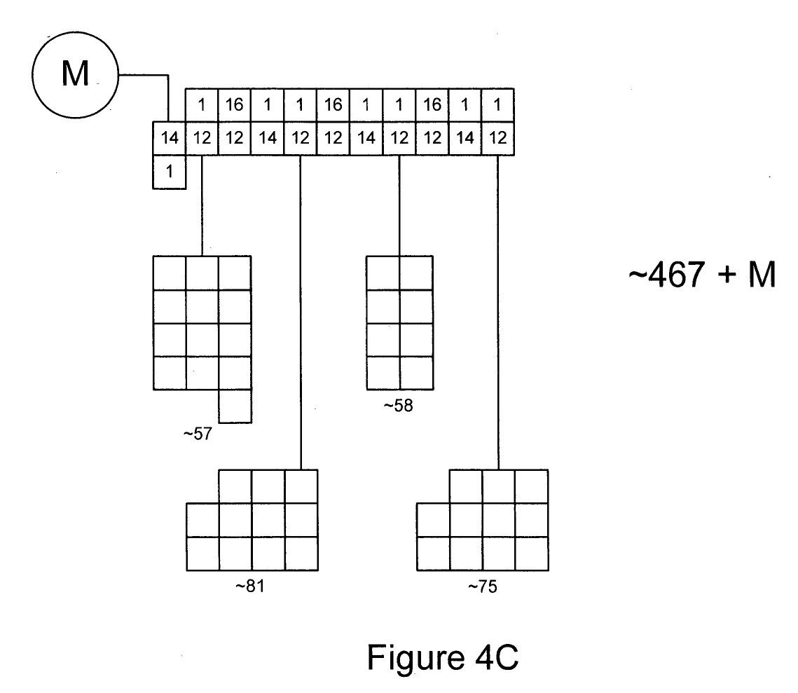

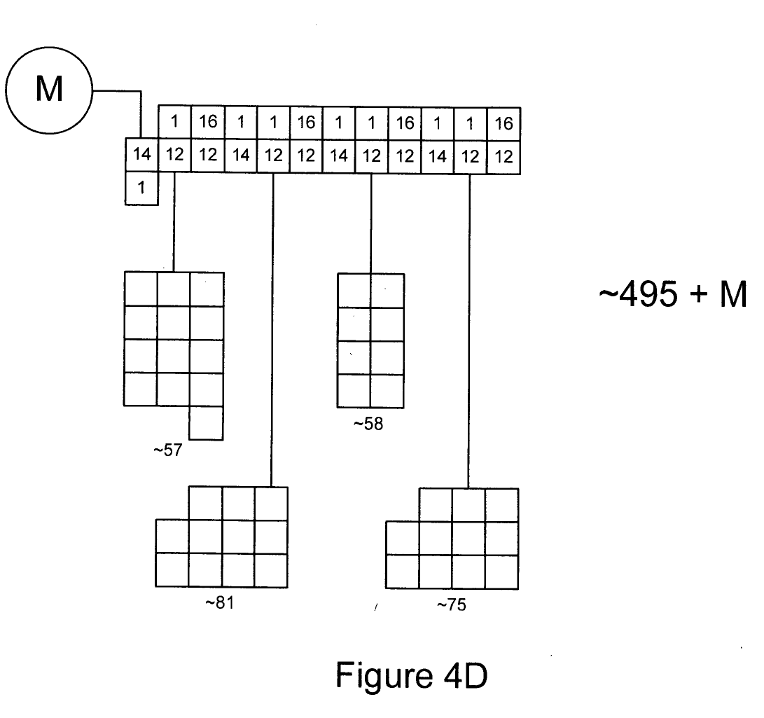

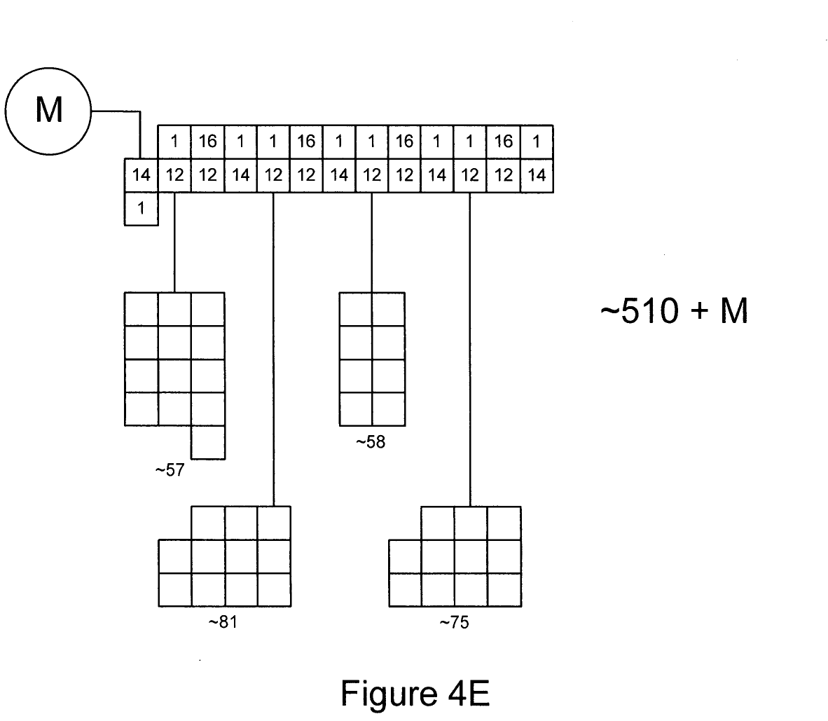

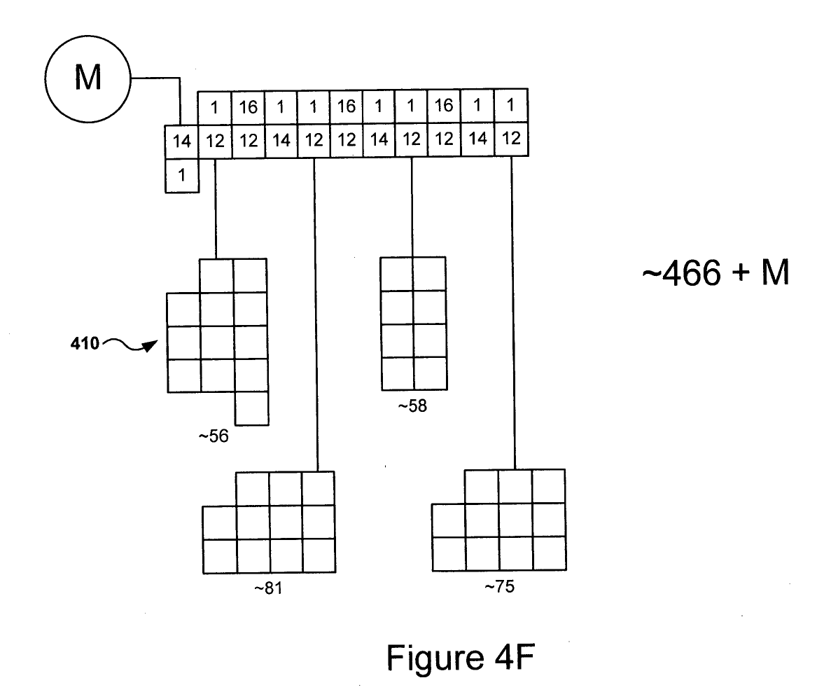

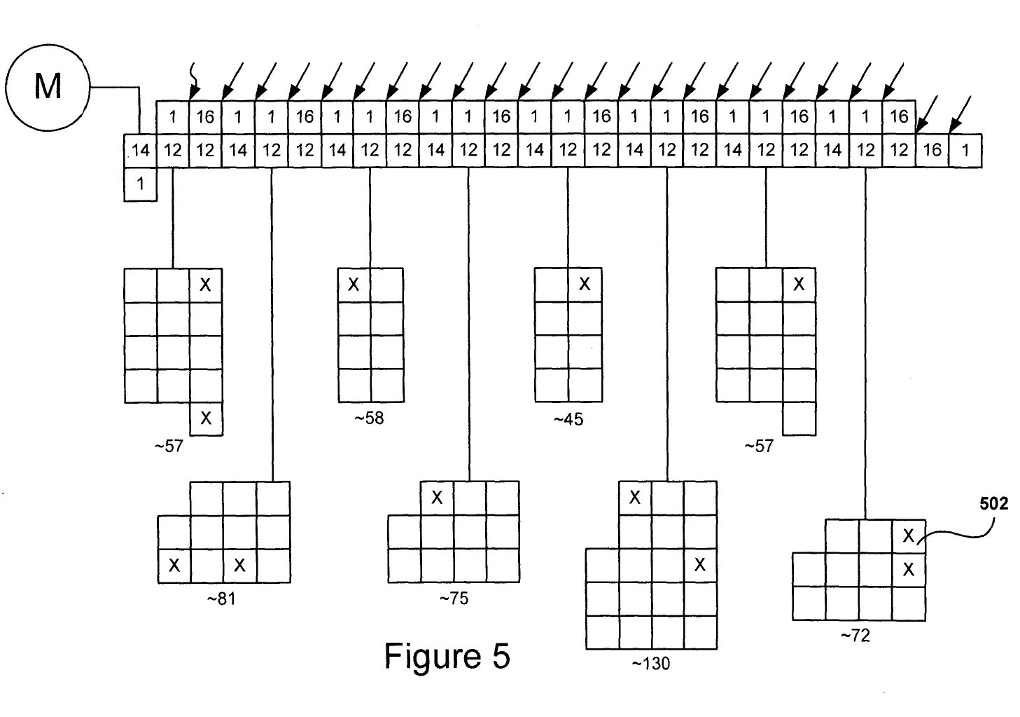

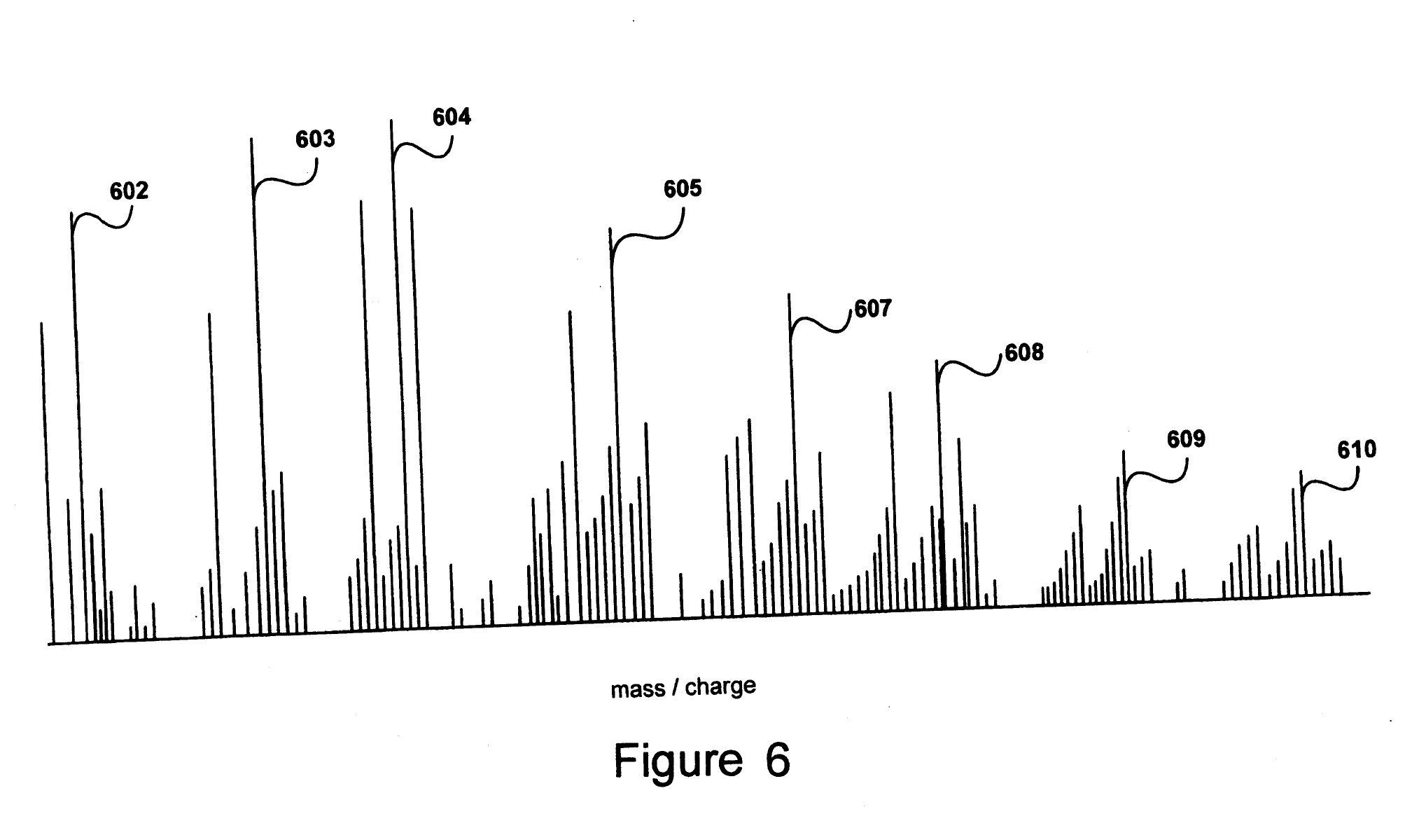

### DETAILED DESCRIPTION OF THE INVENTION

The present invention is related to computational techniques, and systems employing the computational techniques, for determining the subunit sequence of a mass-tagged biopolymer by means of mass spectrometry. Currently, extremely computationally intensive methods are employed to analyze a mass spectrum produced by ionizing, vaporizing and fragmenting the mass-tagged biopolymer by ranking each possible biopolymer-fragment subunit sequence of a known length or range of lengths produced the mass spectrum. Even in the case of a relatively short sequencing of a protein, such as a 7-subunit sequence prediction task, assuming that any of the 20 common amino acids may occur at each position within the subunit sequence, a total of 20⁷, or 1.28 billion different subunit sequences are possible. In the case of mass spectrometry of proteins, two of the 20 common amino-acid subunits have identical masses, and therefore subunit sequences determined by mass spectrometry involve 19, rather than 20, characterizable amino acids, with the resulting subunit sequences ambiguous with respect to the occurrence of the two amino acids with identical masses. Thus, in using mass spectrometry to determine the 7-subunit sequence of a protein, 19⁷, or approximately 894,000,000 different unique subunit sequences are evaluated and ranked by currently available methods, and the greatest ranked sequence is deemed the most probable sequence of the 894,000,000 different subunit sequences and is selected as the 7-subunit sequence of the protein from which the mass spectrum was produced. In alternative embodiments, the ranking may be related to a quantity other than probability, and embodiments of the method of the present invention may be employed to find a sequence optimal with regard to other measurable, calculable, or estimable quantities or characteristics.

Estimating the most probable subunit sequence involves calculating a sequence ranking for each subunit sequence with respect to the mass spectrum. In particular, when comparing two sequences, the sequence with the greater sequence ranking is considered to be the more likely sequence to have produced the mass spectrum under analysis. An important component of the ranking calculation is provided by a computer routine “MS”. The “MS” routine is called with an argument specifying the mass or, equivalently, the subunit-sequence identity of a biopolymer fragment to evaluate the probability that an ion of the specified mass, or the mass corresponding to the specified subunit-sequence, was present in the ionized and vaporized fragments from which the mass spectrum was produced. The function “MS” is a computationally intensive function that returns a score reflective of the probability that the fragment of specified mass or subunit sequence was present in the sample. As noted before, the mass spectrum may contain hundreds or thousands of peaks, and may be affected by isotopes, non-labeled fragments, sample contamination, impure samples, instrumental error, and other experimental errors. Consequently, producing a score reflective of the probability that a particular ion of a particular mass contributed peaks to the mass spectrum is a computationally intensive task.

The overall ranking assigned to a particular subunit sequence is reflective of the probability that the particular subunit sequence is that of the original biopolymer subjected to mass spectrometry analysis. The overall ranking for a particular subunit sequence is determined as a function of the MS scores of possible prefix subsequences of the particular subunit sequence as well as the MS score of the particular subunit sequence. In this context, the term “prefix subsequence” refers to a non-null subsequence that begins with the first subunit of the biopolymer-fragment subunit sequence, and that therefore includes the mass tag. Thus, for example, for a 6-subunit biopolymer fragment with subunits R₁, R₂, R₃, R₄, R₅, R₆, where Rₓ represents the identity of the subunit at sequence position x, the overall sequence ranking for the 6-subunit biopolymer is a function of the scores returned by the following calls to the function “MS:”

MS (M_{R}_{₁})
MS (M_{R}_{₁} + M_{R}_{₂})
MS (M_{R}_{₁} + M_{R}_{₂} + M_{R}_{₃})
MS (M_{R}_{₁} + M_{R}_{₂} + M_{R}_{₃} + M_{R}_{₄})
MS (M_{R}_{₁} + M_{R}_{₂} + M_{R}_{₃} + M_{R}_{₄} + M_{R}_{₅})
MS (M_{R}_{₁} + M_{R}_{₂} + M_{R}_{₃} + M_{R}_{₄} + M_{R}_{₅} + M_{R}_{₆})

where M_{R}_{ₓ}=mass of subunit Rₓ

Equivalently, as discussed above, the identity of the subunits may be specified to a modified function “MS” which accepts a variable number of arguments and uses a table of subunit masses in order to determine the mass of the specified sequence or subsequence. With the modified function “MS”, the overall ranking of the biopolymer-fragment subunit sequence R₁, R₂, R₃, R₄, R₅, R₆ can be expressed as a function of the following six scores returned by six calls to the function “MS:”

MS (R₁)
MS (R₁, R₂)
MS (R₁, R₂, R₃)
MS (R₁, R₂, R₃, R₄)
MS (R₁, R₂, R₃, R₄, R₅)
MS (R₁, R₂, R₃, R₄, R₅, R₆)

Of course, in either form of the function “MS,” the subunit masses employed for internal subunits of the biopolymer need to be the masses of subunits incorporated within a biopolymer, or, in other words, the masses of atoms lost in the condensation reactions need to be subtracted from the monomer masses. Similarly, the masses of the terminal subunits need to be properly computed. In addition, in either form of the function “MS”, the presence of the mass tag is assumed and its mass is intrinsically included.

The overall ranking of a particular sequence can be computed by combining the individual scores returned by the function “MS” in different ways, including summing the individual scores, multiplying the individual scores together, or employing more complex operations. For the purposes of describing one embodiment of the present invention, the following overall ranking function F for a biopolymer fragment containing p subunits, with each subunit being one of n different possible subunit types, is provided as follows:

F (R₁, R₂, . . . Rₚ) =

MS (R₁) +
MS (R₁, R₂) +
MS (R₁, R₂, R₃) +

.
.
.

MS (R₁, R₂, . . . Rₚ)

where Rₓ=1, 2, 3 . . . n.

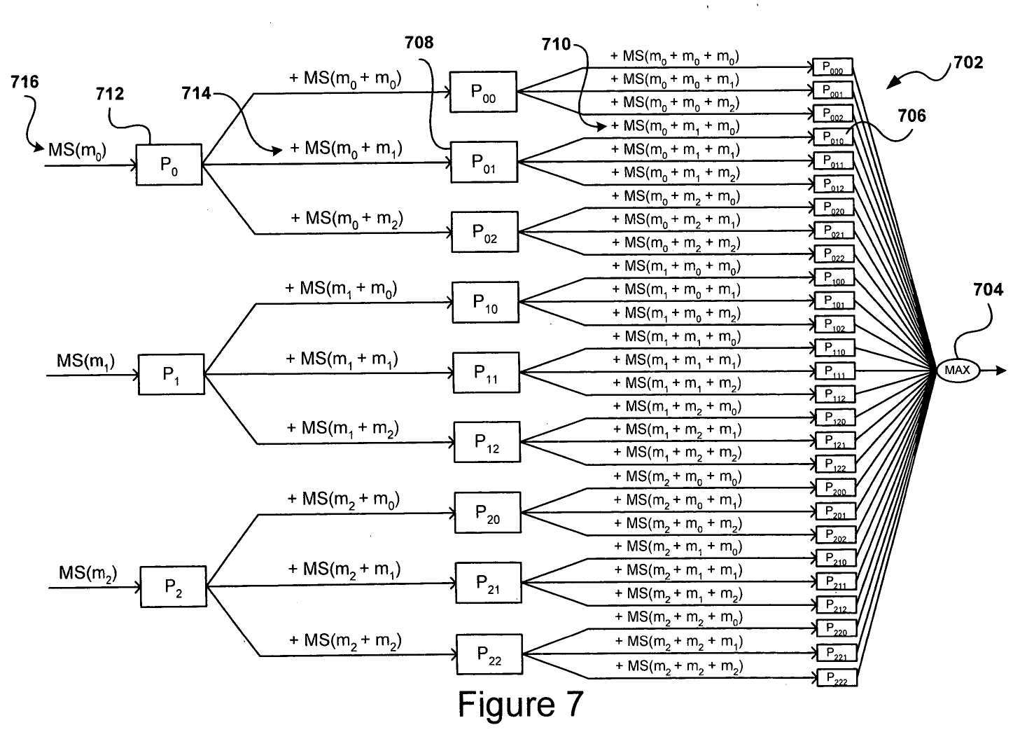

FIG. 7 illustrates a currently available process by which a greatest ranked 3-subunit sequence of a biopolymer is determined from the mass spectrum produced by mass tagging the biopolymer and subjecting the biopolymer to mass spectrometry. In the example shown in FIG. 7, the subunits of the biopolymer may each be one of three different possible types of subunit, specified by the integer values “0,” “1,” and “2.” There are therefore 3³, or 27, different possible 3-subunit biopolymer sequences in this example, shown in column 702 of FIG. 7. Note that the notation “P_{xyz}” stands for the sequence ranking F(x,y,z) of the sample biopolymer fragment of subunit sequence x, y, z, where x, y, and z are elements of the set {0,1,2}. Since the P_{xyz} value depends on the order of the subunits x, y, and z, P_{xyz} can be thought of as a sequence permutation ranking. As mentioned before, the P_{xyz} value reflects the probability that a sample biopolymer fragment of subunit sequence x, y, z contributed peaks to a particular mass spectrum. As can be seen in FIG. 7, the process of determining the most probable biopolymer-fragment subunit sequence involves computing the sequence ranking of each possible biopolymer-fragment sequence P_{xyz} and then selecting the sequence with the greatest sequence ranking. The selection of the sequence with greatest sequence ranking is indicated by the circle 704 in FIG. 7. The sequence ranking of each subunit sequence is determined by summing the “MS” sequence scores of all the prefix subsequences of the sequence and the “MS” score for the entire sequence. Thus, for example, the sequence ranking assigned to sequence “0,1,0” 706, P₀₁₀, equals the sequence ranking of subsequence “01” 708, P₀₁, added to the score returned by the function “MS” for sequence “0,1,0” 710. The sequence ranking assigned to subsequence “0,1” equals the sequence ranking assigned to subsequence “0” 712, P₀, added to the score returned by function “MS” for subsequence “0,1” 714. The sequence ranking for subsequence “0” 712 is simply the score returned by function “MS” for the subsequence “0” 716. The total number of calls to function “MS” in order to determine the most probable 3-subunit subsequence, as illustrated in FIG. 7, is:

$${\sum }_{i = 1}^{p} {n}^{i}$$

where p is the number of subunits and n is the number of different subunit types.

Thus, in the case illustrated in FIG. 7, 3+3²+3³, or 39, different calls to function “MS” are required in order to determine the most probably 3-subunit sequence. Clearly, as the number of different possible subunit types n and the number of sequenced subunits within a polymer p increase, the number of calls to function “MS” grows to immense sizes. For example, 49,659,540 calls to “MS” are needed in order to evaluate the most probable 6-subunit sequence of a protein using the currently available permutations-based method illustrated in FIG. 7. Note that, in column 702, the sequence rankings of each permutation of 3 subunits, each having one of 3 different subunit types, have been calculated.

An important observation that motivated the more computationally efficient method of the present invention is that an identical score is produced by the function “MS” for any permutation of a set of subunit types, or subunit-type combination. In other words, in the case of a 3-subunit biopolymer fragment that includes subunits of types x, y, and z:

MS (x, y, z)=MS (x, z, y)=MS (y, x, z)=MS (y, z, x)=MS (z, x, y)=MS (z, y, x)

Rather than computing individual sequence rankings for each possible sequence via nested calls to function “MS,” as shown in FIG. 7, it is instead possible to calculate the maximum sequence ranking of the sequence permutations of a particular combination of subunit types. Thus, the set of possible sequence permutations can be partitioned into a set of possible subunit-type combinations, and a maximum sequence ranking for each subunit-type combination can then be calculated. This calculated value is referred to as the “maximum sequence ranking” of the combination, or simply referred to as the “combination ranking.”

Note that, just as a polymer sequence can have subunits with duplicated subunit types, the subunit combination described here permits duplicated subunit types within the combination. This is often referred to as “combinations with replacement” in mathematical literature. While a combination without replacement can be thought of as an unordered set of elements selected from a larger set of elements, a combination with replacement is commonly referred to as a “bag” or “multiset” to clarify that duplications are permitted. In other words, the use of the term “subunit combination” in these descriptions refers more precisely to a bag of subunits, wherein multiple subunits may be of the same type. Although the subunits of a subunit combination are unordered, these descriptions impose a representational order to facilitate the description of the methods of the present invention. Finally, the number of subunits in a combination is referred to as the length of a combination.

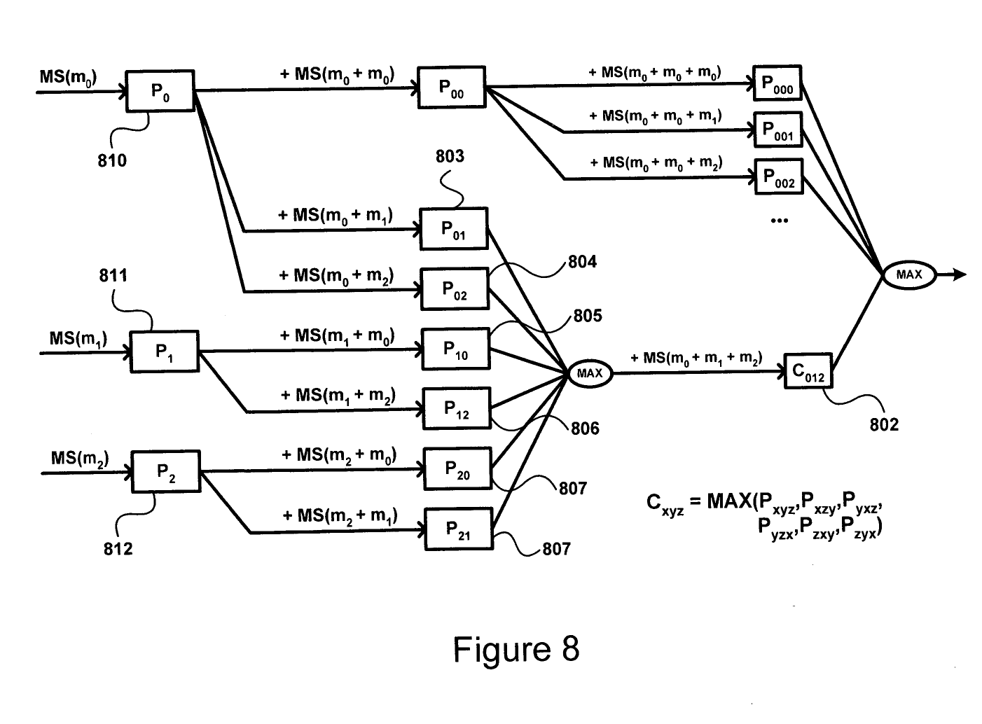

FIG. 8 illustrates calculation of the maximum sequence ranking of a set of sequences representing the permutations of a particular combination of subunit types. In FIG. 8, the maximum sequence ranking for the set of subunit sequences {“0,1,2”, “0,2,1”, “1,0,2”, “1,2,0”, “2,0,1”, “2,1,0”} 802, C₀₁₂, is the greatest sequence ranking assigned to any of the possible 2-subunit subsequences “0,1” 803, “0,2” 804, “1,0” 805, “1,2” 806, “2,0” 807, and “2,1” 808 added to the score returned by the function “MS” for the sequence “012.” In other words, the value C₀₁₂ 802 in FIG. 8 represents the maximum sequence ranking value for any permutation of the subunit combination “0,1,2.” Similarly, in general, the combination ranking value C_{xyz} is the maximum sequence ranking value for any sequence that is a permutation of the subunit combination “x,y,z.” In the following, a subunit combination is referred to as a “combination,” and the string of integers representing the types of subunits is referred to as a “combination-identifier string” (“CID”). CIDs, in the described embodiment, are always written with subunit-type-specifying integers in ascending order.

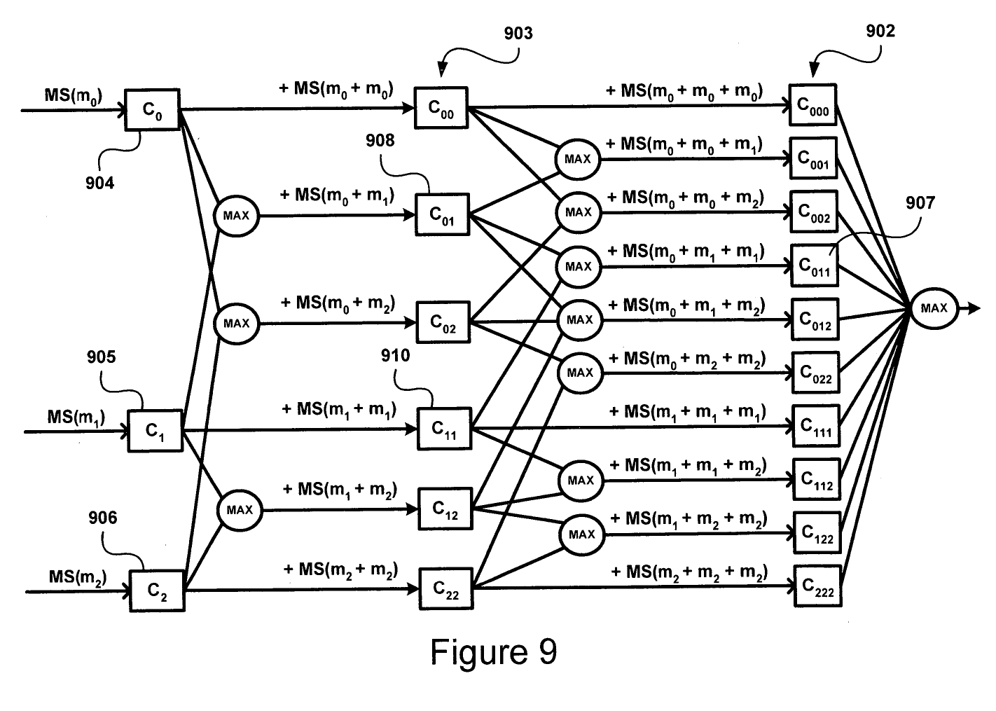

The combination-based approach illustrated in FIG. 8 leads to a vastly more efficient computation of the most probable (i.e. greatest ranked) sequence. FIG. 9 illustrates the combination approach to calculating the most probable biopolymer sequence from a mass spectrum that represents one embodiment of the present invention. As shown in FIG. 9, the total number of different possible sequences, representing all permutations of all possible combinations of three subunits, is partitioned into the ten possible combinations of subunit types, shown in FIG. 9 in column 902. The maximum sequence ranking for any sequence representing a permutation of a particular combination is calculated for the combination by adding the maximum sequence ranking of all immediate subcombinations of the combination, where an immediate subcombination is a combination of a number of subunit identities one less than the number of subunit identities of the combination, and then adding the score produced by function “MS” for any single permutation within the combination. This technique is recursively applied back to combinations of single subunits, Cₓ, which have maximum sequence rankings equal to the “MS” scores of their individual single-subunit sequences. Thus, for example, the single-subunit sequence rankings 810-812 in FIG. 8 are equal to the maximum sequence rankings of single-subunit combinations 904-906 in FIG. 9.

To be clear, the sequence combination “0,1,2” includes the permutations, and thus sequences, “0,1,2,” “0,2,1,” “1,0,2,” “1,2,0,” “2,0,1,” and “2,1,0.” The sequence ranking for combination “0,1,2,” C₀₁₂, is equal to the maximum sequence ranking assigned to one of the sequences “0,1,2,” “0,2,1,” “1,0,2,” “1,2,0,” “2,0,1,” and “2,1,0,” or, in other words, C₀₁₂=max(P₀₁₂, P₀₂₁, P₁₀₂, P₁₂₀, P₂₁₀, P₂₀₁) Note that the max( ) function and the term “maximum,” as used in these descriptions, refer to selecting the greatest value. Furthermore, greatest is a term meant to identify the numeric ranking associated with the optimal sequence, where, in certain embodiments, the optimal sequence is the most probable sequence. This might typically be synonymous with the phrase “most positive”. However, other definitions of the function “MS” could be contemplated, for example one in which the indicated presence of a particular fragment in a mass spectrum is reflected by a more negative return value of function “MS”. In that case, “greatest” would be synonymous with “most negative”.

By calculating the maximum sequence ranking for combinations, as shown in FIG. 9, the function “MS” needs to be called far fewer times. In general, to calculate the maximum sequence ranking for combinations of length up to p for a biopolymer, where each subunit can be one of n different types of subunits, the function “MS” needs to be called a number of times equal to:

$${\sum }_{i = 1}^{p} \frac{( n + p - 1 ) !}{p ! ( n - 1 ) !}$$

In the present case, only 19 calls to the function “MS” are made in order to determine the maximum sequence ranking for each possible combination of subunits, as shown in FIG. 9. This is in contrast to the 39 calls to function “MS” employed to calculate the sequence ranking for each different possible sequence permutation, as shown in FIG. 7. As the values of n and p increase, the savings in calls to MS dramatically increase. For example, in the case of a polypeptide sequence of length 6, the combination approach that represents one embodiment of the present invention, shown in FIG. 9, results in only 177,099 calls to the function “MS” as opposed to the 49,659,540 calls made to the function “MS” in the currently available, permutation-based technique shown in FIG. 7.

Once the maximum sequence rankings for all combinations of subunits are determined, as shown in FIG. 9, the greatest ranked sequence permutation is easily obtained. The process involves traversing the hierarchy of combination rankings backwards, from the nodes at level 3 (column 902 in FIG. 9) through the nodes at level 2 (column 903 in FIG. 9) to the first level that contains single-subunit combinations, such as single-subunit combination 904. In the case of example FIG. 9, one would first choose the greatest combination ranking from amongst the combination rankings in column 902. Assuming that the greatest ranking for a combination is the ranking for combination “0,1,1,” C₀₁₁, 907, one would select as the starting node for the backward traversal node 907. One would then proceed to traverse the graph to the left, choosing as each next-lowest-level node the next-lowest-level node that has the greatest combination ranking of all next-lowest-level nodes that represent immediate subcombinations of the currently selected combination. There are only two immediate subcombinations, “0,1” 908 and “1,1” 910, of combination “0,1,1.” Assuming that subcombination “1,1” 910 has the largest combination ranking, node 910 would be selected and the graph traversed again leftward, with the node representing a subcombination of subcombination “1,1” having the largest combination ranking next selected. In the case of the example FIG. 9, there is only one immediate subcombination of subcombination “1,1,” namely subcombination “1” 905. The greatest ranked sequence permutation is the sequence comprising the subunit type identifiers omitted from the CIDs of higher level nodes in traversing to lower level nodes, with the first omitted subunit identifier occupying the final position of the greatest ranked sequence, and each successive omitted subunit identifier occupying successively lower-order positions.

For example, consider the above-described traversal, proceeding right to left through the graph. Starting with node 907, the subunit type identifier omitted from the CID “011” in traversing to node 910, with CID “11,” is “0,” and that is selected as the subunit identifier of the final, or third, subunit of the greatest ranked biopolymer sequence. Proceeding to traverse from node 910 to node 905, the subunit identifier “1” is the subunit identifier omitted from CID “11” to produce CID “1,” and “1” is therefore selected as the identity of the second subunit of the greatest ranked biopolymer sequence. Continuing to node 905, there is only one subunit identifier in the combination, namely “1,” and that is selected as the subunit identity of the first subunit of the greatest ranked biopolymer sequence. Thus, the greatest ranked (i.e. most probable) polymer sequence is “1,1,0.” Note that, in the above-described embodiments, the subunit identifiers are single-digit integers, but they may be multi-digit integers, as in the case of biopolymers with more than 10 subunit types, such as proteins.

A simple, C++-like pseudocode implementation of both the currently available technique, illustrated in FIG. 7, and the combination-based technique of the present invention, one embodiment of which is illustrated in FIG. 9, are next provided. It should be noted that there are a practically limitless number of different ways to implement both the currently available technique and the present invention, and the pseudocode implementations provided below are intended to further illustrate the above-described techniques in general, rather than suggest any particularly favored or most desirable implementation.

First, a number of enumerations and constant declarations are provided:

1 enum biopolymer {DNA, RNA, protein};

2 enum modifications {methylated, formylated, hydroxyprolineContaining};

3 const int maxMod=10;

4 const int maxResidue=15;

5 const int maxMonomer=20;

6 const int maxTable=20000;

The enumeration “biopolymer” allows for specification of the type of biopolymer to be analyzed. The enumeration “modifications” allows various modifications of the basic biopolymer to be provided. Only a few of the possible modifications are shown in the pseudocode. Additional types of parameters may be employed, including parameters specifying the pH of the solution containing the biopolymer, the chemical identity of any buffers or other components of the solution containing the biopolymer, and other such information. The constant “maxMod” represents the maximum number of possible modifications that can be specified for a particular analysis. The constant “maxResidue” represents the maximum value of p, or, in other words, the maximum length of the biopolymer subunit sequence produced by the analysis. The constant “maxMonomer” represents the maximum number of different types of subunits that can be present in a polymer to be analyzed. The constant “maxTable” is an upper limit on the length of a table used to store the combination rankings, denoted Cₓ, C_{xy}, and C_{xyz} in FIG. 9, where x,y,z ε {0, 1, 2} in FIG. 9.

Next, the class “experimentalInfo” is declared:

| | |
|---|---|
| 1 | class experimentalInfo |
| 2 | { |

| | |
|---|---|
| 3 | private: |

| | |
|---|---|
| 4 | double massTagMass; |
| 5 | biopolymer pType; |
| 6 | modifications mods[maxMod]; |

| | |
|---|---|
| 7 | public: |

| | |
|---|---|
| 8 | void addMod(modifications m); |
| 9 | experimentalInfo(double mTag, biopolymer p); |

| | |
|---|---|
| 10 | }; |

The class “experimentalInfo” allows a user to specify and characterize the particular type of mass-tagged biopolymer from which a mass spectrum was produced, and for which the sequence is desired. This class is essentially a stub container for the information that needs to be provided to the function “MS” for that function to properly analyze a mass spectrum, and can be considered to also contain the mass spectrum, or a reference to the mass spectrum. No implementation is provided for this class, since such detail is outside the scope of the present invention.

Next, a declaration for the class “massSpectrumAnalyzer is provided:

| | |
|---|---|
| 1 | class massSpectrumAnalyzer |
| 2 | { |

| | |
|---|---|
| 3 | private: |

| | |
|---|---|
| 4 | int nextC; |
| 5 | int* CIDptr; |
| 6 | int* nxtS; |
| 7 | int* eCIDptr; |
| 8 | int index[maxMonomer][maxResidue]; |
| 9 | int combinationTable[maxTable]; |
| 10 | int levels; |
| 11 | int initIndex(int n, int p); |
| 12 | int CIDFromTableIndex(int tableIndex, int* cid); |
| 13 | int combinationTableIndex(int level, int* cid); |
| 14 | void firstSubCombination(int level, int *cid, int*res); |
| 15 | bool nextSubCombination(int*res); |
| 16 | |

| | |
|---|---|
| 17 | public: |

| | |
|---|---|
| 18 | int numMonomers( ); |
| 19 | char* monomer(int i); |
| 20 | int MS(int curNum, int* i); |
| 21 | bool newMethod(int p, int* residues); |
| 22 | bool iterativeOldMethod(int p, int* residues); |
| 23 | int recursiveOldMethod(int ranking, int hiRanking, |

| | |
|---|---|
| 24 | int i, int p, int* sequence, |
| 25 | int* residues); |

| | |
|---|---|
| 26 | massSpectrumAnalyzer(experimentalInfo e); |

| | |
|---|---|
| 27 | }; |

The class “massSpectrumAnalyzer” includes member functions that implement the currently available technique, illustrated in FIG. 7, and the technique of the present invention, one embodiment of which is illustrated in FIG. 9. The class “massSpectrumAnalyzer” also includes the function member “MS” and a few accessory function members that support the public function members that implement the methods illustrated in FIGS. 7-9. The class “massSpectrumAnalyzer” is the only pseudocode class for which implementations are provided. The class “massSpectrumAnalyzer” includes the following private data members, declared above on lines 4-10: (1) nextC, a variable including the subunit identifier in the position within a CID to be skipped in extracting a next subcombination's CID: (2) CIDptr, nxtS, and eCIDptr, three integer pointers that point into an integer array representing a CID; (3) index, a two-dimensional array containing an index table, to be described below; (4) combinationTable, an array that contains all of the combination nodes, such as combination node 907 in FIG. 9, computed by the method illustrated in FIG. 9; and (5) levels, an integer that stores the number of levels of combination nodes or, in other words, the number of subunits in a polymer sequence p. The class “massSpectrumAnalyzer” includes the following private function members, declared above on lines 11-15: (1) initIndex, a function member that initializes the two-dimensional array “index” declared above on line 8; (2) CIDFromTableIndex, a function member that calculates the CID corresponding to a cell in the array “combinationTable,” declared above on line 9; (3) combinationTableIndex, a function member that calculates the index of a cell in the array “combinationTable” from a CID; (4) firstSubCombination, a function member that computes the first subcombination from a CID; and (5) nextSubCombination, a function member that extracts the next subcombination from a CID. The class “massSpectrumAnalyzer” includes the following public function members, declared above on lines 18-26: (1) numMonomers, a function member that returns the number of different subunit types n; (2) the function “MS,” described above, for which an implementation is not provided; (3) newMethod, a function member that implements an embodiment of the present invention, described above with reference to FIG. 9 (4) iterativeOldMethod, a function member that implements the technique described above with reference to FIG. 7, (5) recursiveOldMethod, a function member that also implements the technique described with reference to FIG. 7, but in a recursive fashion; and (6) a constructor, for which an implementation is not provided.

Next, implementations of various function members are provided below. First, an implementation of the member function “iterativeOldMethod” is provided. This member function implements the currently available technique illustrated in FIG. 7. It is provided to further describe the currently available technique in a relatively concise fashion.

| | |
|---|---|
| 1 | bool massSpectrumAnalyzer::iterativeOldMethod(int p, int* residues) |
| 2 | { |

| | |
|---|---|
| 3 | Int i, j; |
| 4 | int n = numMonomers( ); |
| 5 | Int tRanking, hiRanking = 0; |
| 6 | int score[maxResidue]; |
| 7 | int sequence[maxResidue]; |
| 8 | |
| 9 | if (p > maxResidue) return false; |
| 10 | |
| 11 | for (i = 0; i < p; i++) |
| 12 | { |

| | |
|---|---|
| 13 | sequence[i] = residues[i] = 0; |
| 14 | score[i] = MS(i + 1, sequence); |
| 15 | hiRanking += score[i]; |

| | |
|---|---|
| 16 | } |
| 17 | |
| 18 | i = p − 1; |
| 19 | |
| 20 | while (true) |
| 21 | { |

| | |
|---|---|
| 22 | if (i < 0) break; |
| 23 | sequence[i]++; |
| 24 | if (sequence[i] = = n) |
| 25 | { |

| | |
|---|---|
| 26 | sequence[i− −] = −1; |
| 27 | continue; |

| | |
|---|---|
| 28 | } |
| 29 | score[i] = MS(i + 1, sequence); |
| 30 | if(I = = p − 1) |
| 31 | { |

| 32 | tRanking = 0; | |
|---|---|---|
| 33 | for (j = 0; j < p; j++) tRanking+= score[j]; | |
| 34 | if (tRanking > hiRanking) | |
| 35 | { | |
| 36 | | hiRanking = tRanking; |
| 37 | | for (j = 0; j < p; j++) residues[j] = sequence[j]; |
| 38 | } | |

| | |
|---|---|
| 39 | } |
| 40 | else i++; |

| | |
|---|---|
| 41 | } |
| 42 | return true; |

| | |
|---|---|
| 43 | } |

The member function “iterativeOldMethod” employs the following local variables, declared above on lines 3-7: (1) i and j, loop control variables; (2) n, the number of different types of subunits in the polymer; tRanking and hiRanking, variables that hold ranking values computed for the currently considered and highest ranked polymer sequence; (4) score, an array containing the scores returned by the function “MS” for the currently considered polymer sequence and prefix subsequences; and (5) sequence, an array of integers that specifies the subunit sequence for the currently considered polymer sequence. In the for-loop of lines 11-16, an initially considered sequence and scores returned by the function MS for prefix subsequences of the initially considered polymer sequence are stored in arrays “sequence” and “score.” Next, in the while-loop of lines 20-41, all possible sequences for a polymer of length p, where p is supplied as an argument to function member “iterativeOldMethod”, are generated and the rankings for the generated sequences calculated. Each time a generated sequence is evaluated to have a higher sequence ranking than any of the sequences previously generated, the sequence is copied to an output sequence identifier “residues,” a pointer to which is supplied as argument “residues.” In the while-loop of lines 20-41, the variable “i” indexes a position within the sequence. The while loop varies the type of subunit at that position, and then varies all subsequent positions in order to generate all possible sequences. Thus, the sequences are generated in ascending order, viewing the integers that specify the subunit types as digits within a number composed of p digits. During each iteration of the loop, the subunit identifier at position “i” is incremented on line 23. If the subunit identifier is incremented up to the value n, as detected on line 24, then the subunit identifier is set to minus one, on line 26, and the position variable “i” is decremented, so that, in the next iteration of the loop, the subunit identifier one position back is incremented. The array “score” contains the MS-generated scores for all prefix subsequences of the current sequence. When the position variable “i” equals p minus one, as detected on line 30, then all the MS scores for a sequence of length p have been generated, allowing the overall sequence ranking for the sequence to be evaluated in the for-loop of line 33. The ranking is compared to the current value of “hiRanking,” on line 34, and, if the current ranking is greater than the current value of “hiRanking,” the currently considered sequence stored in array “sequence” is copied to the output sequence on line 37.

Next, a recursive version of the currently available technique, discussed above with reference to FIG. 7, is provided. This method would calculate the most probable sequence within the array “residues” when invoked as in recursiveOldMethod(0,−1,0,p,sequence,residues), assuming the “MS” function returns non-negative scores. This recursive version is not further discussed, and is provided only to show an alternate technique for implementing the currently available method:

| | |
|---|---|
| 1 | int massSpectrumAnalyzer::recursiveOldMethod |

(int ranking, int hiRanking, int i,

| | |
|---|---|
| 2 | int p, int* sequence, int* residues) |

| | |
|---|---|
| 3 | { |

| | |
|---|---|
| 4 | int j, k; |
| 5 | Int tRanking; |
| 6 | |
| 7 | if(i < p) |
| 8 | { |

| | |
|---|---|
| 9 | for (j = 0; j < numMonomers( ); j++) |
| 10 | { |

| 11 | sequence[i] = j; |
|---|---|
| 12 | tRanking = ranking + |
| | MS(i + 1 , sequence); |
| 13 | hiRanking = recursiveOldMethod |

(tRanking, hiRanking, i + 1,

| | |
|---|---|
| 14 | p, sequence, residues); |

| | |
|---|---|
| 15 | } |

| | |
|---|---|
| 16 | } |
| 17 | else if (ranking > hiRanking) |
| 18 | { |

| | |
|---|---|
| 19 | for (j = 0; j < i; j++) residues[j] = sequence[j]; |
| 20 | hiRanking = ranking; |

| | |
|---|---|
| 21 | } |
| 22 | return hiRanking; |

| | |
|---|---|
| 23 | } |

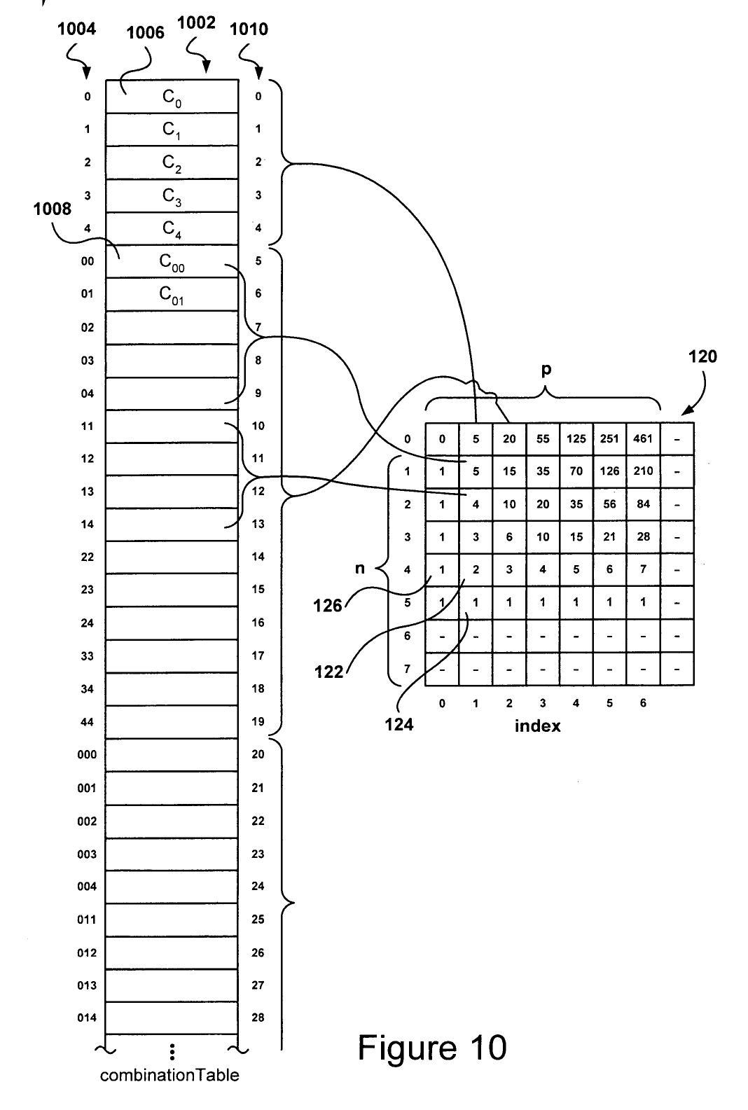

The following six function member implementations together compose a C++-like pseudocode implementation of an embodiment of the present invention, discussed above with respect to FIG. 9. The implementation is described both with respect to the following pseudocode, as well as to FIG. 10. FIG. 10 shows two data structures that enable one embodiment of the method of the present invention to be carried out in a straightforward manner. The first data structure in FIG. 10 is the array “combinationTable” 1002. This array contains the maximum sequence rankings for combinations, or, in other words, the combination nodes, calculated by the method discussed with reference to FIG. 9. In FIG. 10, the left-hand column of indices 1004 indicate the CIDs corresponding to each entry. Thus, for example, the first entry in the combinationTable 1006 includes the value C₀ corresponding to node 904 in FIG. 9. The entry 1008 includes the value C₀₀ corresponding to node 907 in FIG. 9. The right-hand column of indices 1010 in FIG. 10 are the array indices of each cell. Thus, any particular combination maximum sequence ranking value Cₓ . . . can be indexed directly through a sequential array index or via a CID. The two-dimensional table “index” 120 is used to compute CIDs from array indices and to compute array indices from CIDs. In the example used to generate FIG. 10, n=5 and p=7. The contents of the table “index” 120 are related to the values of a Pascal's Triangle, and are easily computed, as described below.

An implementation for the member function “initIndex” is provided below:

| | |
|---|---|
| 1 | int massSpectrumAnalyzer::initIndex(int n, int p) |
| 2 | { |

| | |
|---|---|
| 3 | int i, j, ip, jm; |
| 4 | int total = 0; |
| 5 | |
| 6 | levels = p; |
| 7 | for (i = 1; i <= n; i++) index[i][0] = 1 ; |
| 8 | index[0][0] = 0; |
| 9 | for(j = 1, jm = 0; j < p; j++, jm++) |
| 10 | { |

| | |
|---|---|
| 11 | index[n][j] = 1; |
| 12 | for (i = n − 1, ip = n; i >= 0; i− −, ip− −) |
| 13 | { |

| | |
|---|---|
| 14 | index[i][j] = index[ip][j] + index[i][jm]; |

| | |
|---|---|
| 15 | } |

| | |
|---|---|
| 16 | } |
| 17 | p− −; |
| 18 | for (i = 0; i <= n; i++) total += index[i][p]; |
| 19 | return total; |

| | |
|---|---|
| 20 | } |

This member function computes the values of the table “index” (120 in FIG. 10). This implementation is most easily described with reference to FIG. 10. First, on line 7, the entries in the first column of the table of rows 1 through n are set to one, and the first entry in the first column of the table “index” is set to zero, on line 8. The values of each subsequent column are computed starting from the final row upward. The final value in each column is 1, set on line 11. The next value in each column is the sum of the next lowest value in the column, and the value to the left in the preceding column. For example, the value “2” in the cell with indices (4,1) 122 is the sum of the value “1” in cell (5,1) 124 and the value “1” in cell (4,0) 126. These values have interesting properties. For example, values in row 0 indicate the number of nodes in the previous level. For example, the 0 in cell “0,0” indicates that there are no nodes in the level prior to the first level. The value “5” in cell (0,1) indicates that there are five nodes in the first level of combination nodes.

Next, an implementation for the function member “combinationTableIndex” is provided:

| 1 | int massSpectrumAnalyzer::combinationTableIndex(int level, |
|---|---|
| | int* cid) |
| 2 | { |

| | |
|---|---|
| 3 | Int i, j; |
| 4 | int offset = 0; |
| 5 | |
| 6 | i = 0; |
| 7 | for (j = level; j >= 0; j− −) |
| 8 | { |

| | |
|---|---|
| 9 | while (i <= *cid) |
| 10 | { |

| | |
|---|---|
| 11 | offset += index[i][j]; |
| 12 | i++; |

| | |
|---|---|
| 13 | } |
| 14 | cid++; |

| | |
|---|---|
| 15 | } |
| 16 | return offset; |

| | |
|---|---|
| 17 | } |

This function member calculates a combinationTable array index corresponding to a CID supplied as argument “cid.” An implementation of this function is best described with reference to FIG. 10. In order to compute the index, one starts at the first entry of the column corresponding to the level of the CID. The level of the CID is one less than the number of subunit identifiers in the CID. The index is computed by summing the values encountered in a traversal of the table “index,” starting with the first entry in the column corresponding to the level of the CID. The first column is traversed, element by element, downward a number of times equal to the value in the first identifier of the CID. Then, the traverse moves leftward, to the preceding column, and continues in similar fashion. When all the entries encountered in the traverse are summed together, the index of the combinationTable entry corresponding to the CID is produced.

Next, an implementation of the member function “CIDFromTableIndex” is provided:

| 1 | int massSpectrumAnalyzer::CIDFromTableIndex(int |
|---|---|
| | tableIndex, int* cid) |
| 2 | { |

| 3 | Int i, j; |
|---|---|
| 4 | int length; |
| 5 | |
| 6 | j = levels − 1; |
| 7 | while (index[0][j] > tableIndex) |
| | j− −; |
| 8 | length = j + 1; |
| 9 | tableIndex = tableIndex − |
| | index[0][j]; |
| 10 | i = 1; |
| 11 | 1 while (j >= 0) |
| 12 | { |

| 13 | while (tableIndex >= |
|---|---|
| | index[i][j]) |
| 14 | { |

| 15 | tableIndex = tableIndex − |
|---|---|
| | index[i][j]; |
| 16 | i++; |

| | |
|---|---|
| 17 | } |
| 18 | *cid++ = I − 1; |
| 19 | j− −; |

| | |
|---|---|
| 20 | } |
| 21 | return length; |

| | |
|---|---|
| 22 | } |

This member function takes a combinationTable array index as input, and outputs the corresponding CID. The process is essentially a traverse of the table “index” in a fashion similar to the traverse described above, with respect to the member function “combinationTableIndex,” with the difference that encountered values are subtracted from the supplied combinationTable array index during the traversal.

Next, implementations for the member functions “firstSubCombination” and “nextSubCombination” are provided:

```
  1 void massSpectrumAnalyzer::firstSubCombination(int level, int *cid
    int*res)
  2 {
  3 int* sweep;
  4
  5 CIDptr = cid;
  6 sweep = cid;
  7 eCIDptr = cid + level;
  8 nxtS = eCIDptr;
  9 nextC = *nxtS;
10
11 while (sweep < eCIDptr) *res++ = *sweep++;
12 }
  1 bool massSpectrumAnalyzer::nextSubCombination(int*res)
  2 {
  3 int* sweep = CIDptr;
  4
  5 while (nxtS >= CIDptr && *nxtS == nextC) nxtS−−;
  6 if (nxtS < CIDptr) return false;
  7 nextC = *nxtS;
  8 while (sweep <= eCIDptr)
  9 {
10 if (sweep != nxtS) *res++ = *sweep;
11 sweep++;
12 }
13 return true;
14 }
```

These member functions extract immediate subcombination CIDs from a supplied CID. Thus, for example, if the CID “112” is supplied, calls to “firstSubCombination” and “nextSubCombination” extract, one by one, the CIDs “11” and “12” from the supplied CID “112.” The immediate subcombinations are easily generated by successively omitting a single subunit identifier from the supplied CID. The first omitted subunit identifier is the final subunit identifier in the supplied CID, and the next omitted subunit identifier is the next lowest-valued subunit identifier encountered in the supplied CID traversing the CID leftward.

Finally, an implementation for one embodiment of the present invention is provided in the following pseudocode implementation of the member function “newMethod:”

```
  1 bool massSpectrumAnalyzer::newMethod(int p, int* residues)
  2 {
  3 int numCnodes;
  4 int i, j;
  5 int cid1[maxResidue + 1];
  6 int cid2[maxResidue + 1];
  7 int level;
  8 int curC, maxC, maxI, maxMaxC, omittedIdentifier;
  9 int *nxt;
10
11 numCnodes = initIndex(numMonomers( ), p);
12 maxI = −1;
13 for (i = 0; i < numCnodes; i++)
14 {
15 level = CIDFromTableIndex(i, cid1) − 1;
16 if (level = = 0)
17 {
18 maxC = 0;
19 }
20 else
21 {
22 firstSubCombination(level, cid1, cid2);
23 maxC = combinationTable[combination
              TableIndex(level − 1, cid2)];
24 while (nextSubCombination(cid2))
25 {
26 curC = combinationTable[combination
                  TableIndex(level − 1, cid2)];
27 if (curC > maxC) maxC = curC;
28 }
29 }
30 combinationTable[i] = maxC + MS(level + 1, cid1);
31 if (level == p−1 && (maxI == −1 || combinationTable[i] >
          maxMaxC))
32 {
33 maxMaxC = combinationTable[i];
34 maxI = i;
35 }
36 }
37 nxt = residues + p − 1;
38 j = p − 1;
39 i = maxI;
40 while (j >= 0)
41 {
42 level = CIDFromTableIndex(i, cid1) − 1;
43 firstSubCombination(level, cid1, cid2);
44 i = maxI = combinationTableIndex(level − 1, cid2);
45 maxC = combinationTable[i];
46 omittedIdentifier = *nxtS;
47 while (nextSubCombination(cid2))
48 {
49 i = combinationTableIndex(level −1, cid2);
50 curC = combinationTable[i];
51 if (curC > maxC)
52 {
53 maxC = curC;
54 maxI = i;
55 omittedIdentifier = *nxtS;
56 }
57 }
58 i = maxI;
59 *nxt−− = omittedIdentifier;
60 j−−;
61 }
62 return true;
63 }
```

The function member “newMethod” uses the following local variables, declared above on lines 3-9: (1) numCnodes, the number of combination nodes generated during the analysis; (2) i and j, loop-control variables; (3) cid1 and cid2, arrays for holding CIDs; (4) level, the current level of the combination-node graph; (5) curC, maxC, maxi, maxMaxC, and omittedIdentifier, variables that hold the combination ranking values, indexes of combination nodes; and the omitted subunit identifier during a leftward traversal, and (6) nxt, an integer pointer into the output sequence “residues.”

On line 11, the table “index” is initialized, and the total number of combination nodes returned and stored in variable “numCnodes.” Next, in line 12, “maxI” is initialized to −1, which forces the proper later initialization of “maxI” and “maxMaxC” on lines 33-34. Then, in the for-loop of lines 13-36, all the rankings for combinations are computed and stored into the table “combinationTable.” While the combination rankings for the last level are computed, the greatest computed ranking for a combination is maintained in local variables “maxMaxC” and “maxI,” with maxI storing the index of the greatest computed combination ranking. The combination rankings are straightforwardly computed in the for-loop of lines 13-36, which iterates sequentially over all entries in the table “combinationTable”. For each table entry, the level and corresponding CID is determined on line 15. If the level is 0 (corresponding to a 1-subunit CID), then the maximum stored combination ranking for any of the immediate subcombinations, “maxC” is set to 0. Otherwise, all the immediate subcombination CIDs are extracted from the computed CID on lines 22-23 and the while-loop of lines 24-28. Then, the maximum stored combination ranking for any of the immediate subcombinations, “maxC”, is added to the value returned by function “MS” for the current combination, and the resulting sum stored as the ranking of the combination on line 30. Once all the combination rankings are computed and stored in the table “combinationTable,” the graph of combination nodes, as shown in FIG. 9, is traversed from right to left, selecting the maximally ranked combination node at each level in the while-loop of lines 40-61. The greatest ranked ordered sequence is obtained by selecting the omitted subunit identifiers in the CIDs of the traversed combination nodes, as explained above.

Finally, a pseudocode main routine is provided to show how the pseudocode implementations may be called.

```
  1 int main(int argc, char* argv[])
  2 {
  3 experimentalInfo e(360.35, protein);
  4 massSpectrumAnalyzer m(e);
  5 int sequence[15];
  6 int tSequence[15];
  7 int sum = 0;
  8
  9 m.iterativeOldMethod(7, sequence);
10 sum = m.recursiveOldMethod(0, −1, 0, 7, tSequence, sequence);
11 m.newMethod(7, sequence);
12
13 return 0;
14 }
```

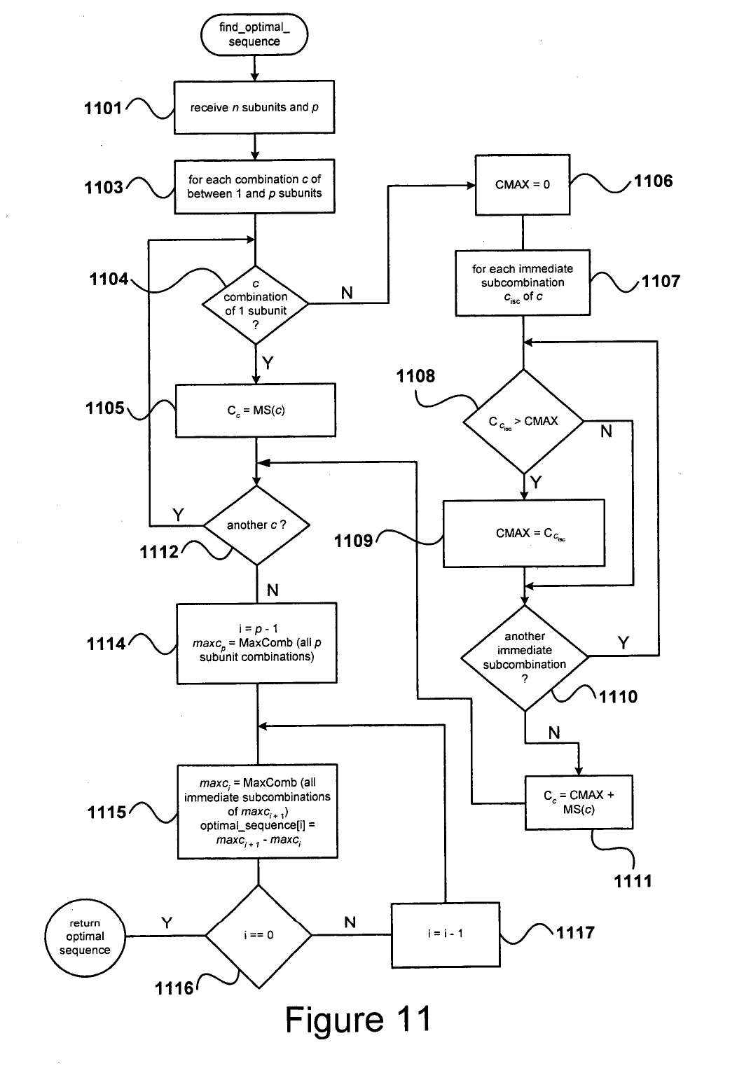

FIG. 11 is a flow-control diagram of a routine “find_optimal_sequence,” an embodiment of the present invention. In step 1101, the routine “find-optimal_sequence” receives specifications of n subunits and the number of subunits p in a desired optimal sequence. Next, in the loop comprising steps 1103-1112, find_optimal_sequence determines the combination ranking C_{c} for every currently considered combination c of between 1 and p subunits. In step 1104, find_optimal_sequence determines whether or not the currently considered combination c is a one-subunit combination. If so, find_optimal_sequence assigns, in step 1105, to the currently considered combination c the score for the single subunit produced by a call to the sequence-scoring function MS. If not, then in the nested loop of steps 1106-1110, find_optimal_sequence determines the maximum ranking CMAX of the immediate subcombinations of the currently considered combination c and, in step 1111, assigns the ranking of the currently considered combination, Cc, to be the sum of the maximum ranking of an immediate subcombination CMAX and the score returned by the function “MS” for a sequence within the currently considered combination, MS(c). Then, in the loop comprising steps 1114-1117, find_optimal_sequence traverses the combinations, starting with the level of p-subunit combinations, and determines the optimal subunit sequence as the subunits omitted during each traverse from a higher level combination to a next lower level combination, the optimal sequence beginning with the last subunit omitted in the final step of the traversal and ending with the first subunit omitted in the first step of the traversal. At each step of the traversal i, the subunit identity of the optimal sequence optimal_sequence[i] is the subunit omitted in traversing from combination maxcᵢ₊₁ to the maximum-ranked immediate subcombination maxcᵢ. This omitted subunit can be viewed as the result of a bag difference of the subunits of the lower level immediate subcombination from the subunits of the higher level combination. The combinations maxcᵢ₊₁ and maxcᵢ are determined by an assumed utility function, “MaxComb”, which accepts a set of combinations as arguments and returns the combination having the greatest ranking. “MaxComb” returns the null combination if passed the empty set as an argument. For a null combination n and any other combination c, c−n=c.

Although the present invention has been described in terms of a particular embodiment, it is not intended that the invention be limited to this embodiment. Modifications within the spirit of the invention will be apparent to those skilled in the art. For example, as noted above, there are almost limitless numbers of different possible implementations of the currently available method, described above with reference to FIG. 7, and various embodiments of the present invention, one of which is described above with reference to FIG. 9. These implementations may be written in any number of different computer languages, using any number of different control structures and modular organizations, employing many different types of data structures. The pseudocode routines, supplied above, are quite general in nature, and can be easily extended to determine most probable polymer sequences of arbitrary lengths, including arbitrary numbers of different types of subunits. The method can be additionally extended to traverse, in recursive fashion, branching polymers. The method of the present invention may be applied to determining optimal sequences in cases other than mass-spectrometry determination of biopolymer sequences. While an iterative embodiment of the method of the present invention is described above, recursive embodiments are also possible. Note that, while the function “MS” returns an integer value in the above pseudocode, other return value types are possible, including for example single-precision floating point and double-precision floating point.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit the invention to the precise forms disclosed. Obviously many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents:

1. A method for searching for an optimal sequence of subunits from among possible sequences of subunits of a given length p, the subunits having subunit types selected from a set of subunit types T of size n, a function F applied to a sequence of subunits to produce a ranking for the sequence of subunits, the ranking produced by the function F for the optimal sequence of subunits greater than rankings obtained by applying the function F to all non-optimal sequences of subunits, the method comprising:
calculating a ranking for each possible subunit combination of length between I and p including combinations with duplicated subunits, a ranking of a combination of subunits being equal to a ranking of a greatest ranked sequence that is one permutation of the combination of subunits; and
determining the optimal subunit sequence from the calculated subunit-combination rankings.

2. The method of claim 1 wherein determining the optimal subunit sequence from the calculated combination rankings further comprises:
identifying as maxcₚ a combination of length p with a greatest combination ranking;
identifying as maxcᵢ, for each i in a range from p−1 down to 1, a subcombination of maxcᵢ₊₁ having a length i with a greatest combination ranking from among subcombinations of maxcᵢ₊₁ having the length i;
selecting, as a subunit for position 0 in the optimal subunit sequence, a single subunit comprising maxc₁; and
selecting, as a subunit for position i in the optimal subunit sequence, for each position i in the range from 1 to p−1, the subunit that, when added to maxcᵢ, produces maxcᵢ₊₁.

3. The method of claim 1 wherein the rankings produced by F are numeric quantities, with a greatest ranking having a most positive absolute value.

4. The method of claim 1 wherein the rankings produced by F are numeric quantities, with a greatest ranking having an absolute value of 0, and lesser rankings having more positive absolute values.

5. The method of claim 1 wherein the rankings produced by F reflect probabilities, with greater rankings reflecting correspondingly greater probabilities.

6. The method of claim 1 wherein calculating the ranking for all subunit combinations of length between 1 and p further comprises:
assigning to each combination of length 1 a ranking equal to a score returned by a scoring function that returns a score for a specified subunit sequence based on one or more of a measurement, calculation or estimation;
for each combination of length i, with i=2 up to p, assigning to the combination a ranking equal to a sum of a greatest ranking associated with any immediate subcombination of length i−1 of the combination of length i and a score returned by the scoring function for a subunit sequence of length i contained in the combination of length i.

7. The method of claim 6 wherein the sequences are biopolymer sequences, wherein the subunits are biopolymer subunits, wherein the subunit types are selected from among possible biopolymer subunit types, and wherein the scoring function returns a score related to the results of a biochemical analysis.

8. The method of claim 7 wherein the biopolymer is selected from among:
a deoxyribonucleic acid;
a modified deoxyribonucleic acid;
a ribonucleic acid;
a modified ribonucleic acid;
a glycoprotein;
a protein;
a modified protein; and
a polysaccharide.

9. The method of claim 7 wherein the scoring function returns a score reflective of a probability that an ion of mass equal to a supplied mass contributed to a particular analytical result, and wherein the optimal sequence of length p is a most probable subunit sequence of length p of a biopolymer contributing to a particular analytical result.

10. The method of claim 7 wherein the result of a biochemical analysis is a mass spectrum produced by a mass-spectrometry procedure.

11. A computer system that produces optimal sequences by the method of claim 1.

12. Instructions encoding a computer program that implements the method of claim 1 stored in a computer-readable memory.

13. A method for determining the subunit sequence of length p of a mass-tagged biopolymer from a mass spectrum of the mass-tagged biopolymer, the method comprising:
providing a function MS that returns a score reflective of a probability that a specified ion contributed to the mass spectrum;
calculating a ranking for each possible subunit combination of length between 1 and p, including combinations with duplicated subunits, a ranking of a combination of subunits equal to a ranking of a greatest ranked sequence that is one permutation of the combination of subunits; and
determining the subunit sequence of a mass-tagged biopolymer by determining a greatest ranked subunit sequence of length p from the greatest ranked combination of length p and a sequence of greatest ranked immediate subcombinations of the greatest ranked combination of length p of monotonically decreasing lengths of from p−1 to 1.

14. The method of claim 13 wherein calculating rankings for each possible subunit combination of length between 1 and p further comprises:
calculating a ranking of a combination of length i by adding a score returned by the function MS applied to a subunit sequence included in the combination of length i to a ranking of a combination of length i−1 contained in the combination of length i and having a greatest ranking of all combinations of length i−1 contained in the combination of length i.

15. The method of claim 13 encoded in computer instructions stored in a computer-readable medium.

16. A subunit sequence, obtained by the method of claim 13, stored in a computer-readable medium.

17. A computer system that determines the subunit sequence of length p of a mass-tagged biopolymrer from a mass spectrum of the mass-tagged biopolymer by the method of claim 13.

18. A method for determining a particular sequence of n entities from among a large number of sequence-of-entity permutations, the method comprising:
providing a function that scores a specified sequence;
determining the set of combinations with replacement of entities having between 1 and n entities;
determining for each combination with replacement of entities with 1 entity a combination ranking determined by applying the function to the entity contained in the combination with replacement;
determining, for each combination with replacement of entities with more than 1 entity a combination ranking determined by applying the function to a sequence of entities representing a permutation of the entities contained in the combination of replacement of entities and adding to a result of the function a greatest ranking obtained for an immediate subcombination of the combination; and
determining the particular sequence from the determined combinations with replacement having greatest rankings.

19. The method of claim 18 wherein determining the particular sequence from the determined combinations with replacement having greatest rankings further includes:
traversing the combinations with replacement from the combinations with replacement having a greatest ranking with n elements to a combination with replacement having a single entity, supplying as next lowest ordered entity within the particular sequence, for each traversal step, an entity representing a set difference between combinations with replacement involved in the traversal step.

20. The method of claim 19 wherein a first combination with replacement during the traversal is selected as the combination with replacement having n entities with a greatest ranking.

21. The method of claim 20 wherein a next combination with replacement during the traversal is selected as a subcombination of a currently considered combination with replacement with 1 less entity having a greatest ranking.
